(19) United States
(12) Patent Application Publication
(10) Pub. No.: US 2004/0001873 A1
(43) Pub. Date: Jan. 1, 2004

(54) Gelled delivery vehicle containing nutritional ingredients

(76) Inventors: Marvin Dimitrios Base, Manhasset, NY (US); Chris Pavlatos, Bayside, NY (US); Angelo Haligiannis, Plandome Manor, NY (US)

Correspondence Address: HOFFMANN & BARON, LLP, 6900 JERICHO TURNPIKE, SYOSSET, NY, 11791, US

(21) Appl. No.: 10/186,430

(22) Filed: Jul. 1, 2002

(51) Int. Cl. A61K 47/00; A61K 38/39; A61K 31/736

(52) U.S. Cl. 424/439; 514/54; 514/21

(57) ABSTRACT

The present invention provides a format for delivering a desired nutritional package in a firm, gelled delivery vehicle to a human host and methods for preparing same. The format contains a nutritional package which includes at least one nutritional ingredient(s) selected for their desired health benefits. The nutritional ingredients include vitamins, minerals, herbs, and probiotics. The format is consumed by a human host to provide a desired health benefit. Desired health benefits include supplementing an individual's diet and weight loss.

### BACKGROUND OF THE INVENTION

The present invention relates to providing selected nutritional ingredients to a human host, and in particular, to a method and means for delivery of aliquots, or packages, of nutritional ingredients.

Concern for health and nutrition in recent years has focused consumers on various ways to ensure consumption of foods appropriate for delivery of a nutritionally balance diet. Nutritional ingredients, such as vitamins, minerals, herbs, and probiotics are known to be critical to the health and well-being of a human. The role of particular nutritional ingredients in promoting certain health benefits is becoming increasingly clear. However, the majority of humans do not receive adequate amounts of proper nutritional ingredients in their daily diet to obtain certain desired health benefits.

The concern for ensuring a full complement of required nutrients is exacerbated when a consumer is undergoing a dietary regimen to lose weight. In the case of weight reduction, it is desirable to reduce intake of unnecessary and nutritionally-empty calories. Thus, the food industry continuously strives to devise methods and means to deliver desired nutritional packages in an organoleptically pleasing format.

Therefore, there is always a need to provide an easy, attractive format for delivery to humans, a nutritional package containing ingredients selected for their desired health benefits.

### SUMMARY OF THE INVENTION

The present invention provides a format for delivering a desired nutritional package to a human host and a method for treating a human host with same. The present invention also includes a method for preparing and delivering the format to a human host.

The format includes a firm, gelled delivery vehicle and a nutritional package which contains nutritional ingredients. The nutritional ingredients are incorporated, and interspersed and suspended in a gelled delivery vehicle.

The nutritional ingredients can include any combination of vitamin(s), mineral(s), herb(s), and/or probiotics. The nutritional ingredients included in a package depend on the intended health benefit.

The vitamin can be any vitamin. Examples of vitamins useful in the present invention include vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, and para-aminobenzoic acid.

The mineral can be any mineral. Examples of minerals useful in the present invention include boron, calcium, chromium, copper, iron, magnesium, manganese, molybdenum, nickel, phosphorus, selenium, silicon, tin, vanadium, and zinc.

The herb can be any herb. Examples of herbs useful in the present invention include alfalfa, althea root, asparagus root, chamomile, garlic, ginger, ginseng, echinacea, ginkgo biloba, valerian root, kelp, Saint John's Wart, yohimbe, and yucca root.

The probiotic can be any probiotic. Examples of probiotics useful in the present invention include Bifidobacteria and Lactobacillus.

The delivery vehicle is a gelling agent. The gelling agent is any gelling agent capable of sustaining a shape and a volume of a continuous matrix capable of maintaining, suspending, and delivering nutrients. The gelling agent can include gelatin, gum, or combinations thereof.

The gelatin can be any gelatin. Typically the gelatin is derived from animals. The gelatin can be unflavored gelatin or flavored gelatin.

Gums useful in the present invention are usually polysaccharides derived from plants or bacteria. Examples of gums include agar agar gum, guar gum, arabic gun, locust bean gum, and xanthan gum.

The format is ingested by a human host to provide enhanced health benefits. The format can be used to enhance health, such as, for example, weight loss or deliver a nutritional supplement.

As a result of the present invention, a nutritional package is provided in a format of a gelled delivery vehicle, which can be ingested by a human to provide a desired health benefit.

For a better understanding of the present invention, together with other and further advantages, reference is made to the following detailed description, taken in conjunction with the accompanying drawings, and its scope will be pointed out in the claims.

### BRIEF DESCRIPTION OF THE DRAWINGS

FIG. 1. is an illustration of a pan used as a mold for providing one format for the nutritional package of the present invention.

FIG. 2. depicts a tray containing serving portions of nutritional packages in accordance with the format of the present invention in two different time-related dosage levels.

### DETAILED DESCRIPTION OF THE INVENTION

“Format” as used herein means the make-up and arrangement, e.g., size, shape, texture, consistency, color, etc., of a comestible product which is consumed by a human host. The format of the present invention includes a nutritional package and a firm, gelled delivery vehicle.

A “nutritional package” is simply a combination of nutritional ingredients which are selected for simultaneous delivery in the format as described herein. The nutritional ingredients are selected for their desired health benefits.

“Nutritional ingredient” as used herein means a substance which has a real and/or perceived beneficial health benefits, and does not have long-term harmful biological effect(s) on the human host. The beneficial health benefits can include, supplementing nutritional deficiencies in one's diet, and promoting weight loss. Nutritional ingredients which can be used for their beneficial health benefits, include, but are not limited to, vitamins, minerals, herbs, and probiotics.

Vitamins are important nutrients in the diet of a human. A vitamin is any organic substance which is typically essential for the normal growth and activity of humans. Vitamins are fat-soluble or water-soluble. The vitamin can be in the form of a solid or a liquid. The vitamins can be obtained from a multivitamin tablet, such as for example, Centrum® manufactured by Wyeth. The vitamin can be any vitamin. Examples of vitamins include, but are not limited to, vitamin A (retinol), B1 (thiamine), B2 (riboflavin), B complex, B6 (pyridoxine), B12 (cobalamin), C (ascorbic acid), D (cholecalciferol), E (tocopherol), F (linoleic acid), G, H (biotin), and K, and choline, folic acid, inositol, niacin, pantothenic acid, and para-aminobenzoic acid.

The specific health benefits of a particular vitamin are known to those skilled in the art. The nutritional package can contain any combination of vitamins. The amounts and combination of vitamins in a nutritional package can be selected for their known and/or desired benefits to humans in general and/or tailored to a particular individual or patient. For example, if the desired health benefit is for weight loss, the nutritional package can contain Vitamin B6.

Minerals are naturally occurring inorganic substances which are typically essential to the nutrition of humans. The mineral can be any mineral. Examples of minerals include, but are not limited to, boron, calcium, chromium, copper, iron, magnesium, manganese, molybdenum, nickel, phosphorus, selenium, silicon, tin, vanadium, and zinc. Substances containing minerals, such as, for example, a multivitamin tablet containing minerals, can be used as the source of the mineral.

The specific health benefits of a particular mineral are known to those skilled in the art. The nutritional package can contain any combination of minerals. The amounts and combination of minerals in the nutritional package can be selected for their known and/or desired benefits to humans in general and/or tailored to a particular individual or patient. For example, if the nutritional package is for weight loss, the nutritional package can contain the mineral vanadium.

Herbs are plants which are typically used for their medicinal properties. The active medicinal component can be from any part of the plant (e.g., stem, leaves, flowers, seeds, and roots). The medicinal component of the herb can be extracted and purified into a liquid or solid form. The extraction and purification of the active, medicinal component of an herb are known to those skilled in the art. It is not necessary to extract and purify the active medicinal component of the herb. For example, fresh or dried herb can be finely chopped and added to the nutritional package. The herb can be any herb. Examples of herbs include, but are not limited to, alfalfa, althea root, asparagus root, chamomile, garlic, ginger, ginseng, echinacea, ginkgo biloba, valerian root, kelp, Saint John's Wart, yohimbe, and yucca root.

The specific health benefits of a particular herb are known to those skilled in the art. The nutritional package can contain any combination of herbs. The amounts and combination of herbs in the nutritional package can be selected for their known and/or desired benefits to humans in general and/or tailored to a particular individual/patient. For example, if the nutritional package is for weight loss, the nutritional package can contain kelp.

Probiotic, as used herein, is typically a microbial supplement which beneficially improves the microbial balance of an individual. For example, probiotics can aid in digestion and help prevent illness by promoting the growth of good bacteria in the digestive tract of the individual. The probiotic can be any probiotic. Examples of probiotics include, but are not limited to Bifidobacteria and Lactobacillus.

The specific health benefits of a particular probiotic are known to those skilled in the art. The nutritional package can contain any combination of probiotics. The amount and combination of probiotics in the nutritional package can be selected for their known and/or desired benefits to humans in general and/or tailored to a particular individual or patient. For example, if the nutritional package is for treating weight loss, the nutritional package can contain the probiotic Lactobacillus acidophilus.

The nutritional ingredients described above can be use alone, or in combination with one another. For example, the nutritional package can contain vitamin(s); vitamin(s) and mineral(s); vitamin(s), mineral(s) and herb(s); vitamin(s), mineral(s), herb(s), and probiotic(s); vitamin(s), herb(s), and probiotic(s); etc. The combination of nutritional ingredients to include in a nutritional package depends on the desired health benefit.

The format of the present invention includes a firm, gelled delivery vehicle. The firm, gelled delivery vehicle is used to provide strength, support, and a firm structure in which to intersperse and suspend the nutritional ingredients. The delivery vehicle is a gelling agent. The gelling agent is any gelling agent capable of sustaining a shape and a volume of a continuous matrix capable of maintaining, suspending, and delivering nutrients. The amount of gelling agent in the format is an amount sufficient to cause gelling, yet provide a firm, flexible, structural support for the nutritional ingredients.

Several prior art references disclose the use of gelatin in foodstuff. For example, U.S. Pat. No. 6,171,632 B1 to Lanter et al. discloses an animal feed gel for consumption by carnivorous animals. The animal feed gel is used as a replacement for an animal's natural food. There is no indication in Lanter et al. to use the animal feed gel for human consumption as a health aid for a desired health benefit.

The recipe for “Cookie Press Jello Cookies” discloses the combination of alimentary ingredients with a fruit flavored gelatin mix. The gelatin in the cookies is used as an ingredient and therefore is not relied on to provide a format for holding and delivering a nutritional package.

PCT application WO 01/22835 discloses the use of gelatin in a layered cereal bar. The gelatin is used in a binder to hold the ingredients of the cereal bar together.

Other references discuss the use of gelatin as part of a composition for consumption by a human. However, these references do not teach or suggest the use of the gelatin as a means for structurally holding and delivering a nutritional package. For example, U.S. Pat. No. 4,070,488 and U.S. Pat. No. 4,268,529 to Davis, discloses the use of gelatin in a nutritive composition as a source of amino acids. Furthermore, the patents to Davis require that the gelatin be used at a low concentration so as to avoid gelling when added to a liquid and cooled.

The prior art references disclose the use of gelatin in combination with nutritional ingredients. However, there is no disclosure or suggestion to use gelatin as a vehicle for delivering a desired nutritional package as in the present invention.

Gelling agents useful in the present invention as a delivery vehicle typically include gelatin and the gums. Gelatin and the gums can be used alone or in combination with one another. Preferably, the gelling agent is gelatin.

Gelatin is a peptide which is derived from denatured collagen. The gelatin can be from any source. Typically, the gelatin is from an animal source, such as, for example, a cow. The gelatin can be unflavored gelatin or flavored gelatin. The flavored gelatin can be any flavor. Examples of flavored gelatin include, but are not limited to, lemon, lime, cherry, strawberry, watermelon, grape, and orange. The gelatin can be used alone, in combination with another gelatin(s), or in combination with gum(s).

Gums are polysaccharides usually derived from plants or bacteria. The gums assist in the structural integrity and setting of the format of the present invention. The gum can be any gum. Examples of gums include, but are not limited to, agar agar gum, guar gum, arabic gum, locust bean gum, and xanthan gum. The gum can be used alone, in combination with another gum(s), or in combination with gelatin(s).

In order to gell the delivery vehicle, a liquid is typically added to the gelling agent. The liquid can be any polar liquid. Polar liquids include, but are not limited to water, fruit juices, and milk. Preferably, the liquid is water. Additional nutrients and flavor can be added to the format by using fruit juices. The fruit juice can be used alone or in combination with other polar liquids. Examples of fruit juices include, but are not limited to, apple juice, grape juice, cranberry juice, orange juice, and grapefruit juice. The liquid is used, in an amount sufficient to dissolve the gelling agent to form a firm, gelled delivery vehicle, which provides strength, support, and a firm structure in which to intersperse and suspend the nutritional ingredients.

The preparation of the format includes selecting at least one nutritional ingredient which provides a desired health benefit to a human host. As stated above, the nutritional ingredients included in a particular nutritional package of a format depends on the desired health benefit. Once the desired nutritional ingredients are determined, the nutritional package is typically mixed with the gelling agent(s). If, for example, a multivitamin tablet containing vitamins and/or minerals is used as the source of the vitamins and minerals, the tablet is crushed into a powder.

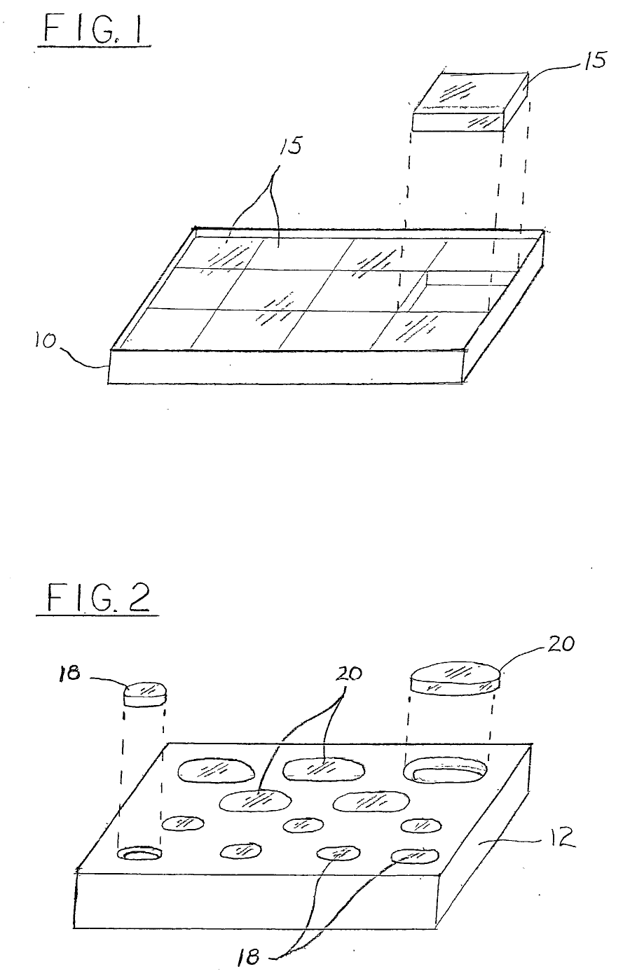

Once the gelling agent is thoroughly mixed with the nutritional package, a liquid is added to dissolve the gelling agent. Measures can be taken to protect heat liable nutritional ingredients, and are known to those skilled in the art. Typically, the liquid used to dissolve the gelling agent is at a warm temperature, at least about 130° F., preferably at least about 140° F., and more preferably at least about 150° F. The solution is stirred until the gelling agent is dissolved. Once the gelling agent is dissolved, the solution can be poured into a mold (see for example, FIG. 1) and allowed to cool. Preferably, the solution is allowed to cool at below room temperature, for example, at refrigeration temperature.

Cooling allows the gelling agent to gell, and results in the incorporation of nutritional ingredient(s), which are interspersed and suspended in the firm, gelled delivery vehicle.

The gelled format, which contains nutritional ingredient(s) interspersed and suspended in a firm, gelled delivery vehicle, can be purchased by a human host. In another embodiment, the nutritional package, containing the gelling agent, can be purchased by a consumer as a powder and formed into a gelled format by, for example, the method described above.

The format can be molded into any shape. Thus, the format can be prepared by molding gelatin with a substantially evenly dispersed nutritional package in a pan 10 (FIG. 1). Once the gel is set, the resulting pan-shaped monolith can be cut into any shape, e.g., rectangle 15. The shape can be a novelty shape, such as, for example, an animal, a geometric shape, or an object. Examples of animal shapes include, but are not limited to, dog, cat, rabbit, lion, giraffe, monkey and elephant. Examples of geometric shapes include, but are not limited to, square, rectangle triangle, star, and circle. Each shape typically represents a serving portion.

It is preferable that the shape be visually pleasing and easily handled by the consumer. For example, if the consumer is a child, the format can be in the shape of an animal, such as a dog.

As stated above, the nutritional ingredients to include in the format of the present invention depends on the desired use of the composition. It is known to those skilled in the art which nutritional ingredient(s) are beneficial for a desired health benefit. The nutritional package can be formulated, for example, to be used as a nutritional supplement to supplement one's diet or to enhance one's health, such as weight loss.

Any human can consume the format of the present invention. The human can be any human which would benefit from ingesting a format containing a desired nutritional package. The human may desire to enhance their health by, for example, losing weight. Ingesting the format results in delivery of a desired nutritional package to the human host.

In another embodiment, the human host can be treated with a nutritional package containing at least one nutritional ingredient selected for its desired health benefit. The nutritional package can also contain two or more nutritional ingredients. The treatment of a human host includes administering to the human, a nutritional package in accordance with a regimen designed to provide a desired health benefit.

The regimen can be any regimen which beneficially results in a desired health benefit for the human host, and can result from a prescription by a physician to obtain a desired health benefit.

The regimen can include a time-related dosage level for administration of the nutritional package. The time-related dosage level includes at least two different dosage levels 18 and 20 (e.g., serving portions) packaged in, for example, a tray 12 (FIG. 2). For example, a smaller dose 18 could be required every four (4) hours, while a larger dose 20 could be required every six (6) hours. The dosage level is any dosage level which results in a desired health effect of a nutritional package, without long-term harmful biological effect(s) on the human host.

The regimen can also be based on frequency of administration, rather than on a strict time schedule. For example, the nutritional package can be formulated for administration of at least once a day, three times a day, etc. The regimen can also be based on time and/or frequency of administration.

The desired health benefit of a nutritional package can be weight loss. The nutritional package for weight loss can be formulated such that the regimen for administration of the package is ingested at least once a day. For example, the human host who desires to lose weight, can ingest the formulated package once a day for two weeks or until the target weight is achieved.

In a preferred embodiment, the regimen for a nutritional package for weight loss can be formulated for ingestion at least three times a day for three days. Without being bound by theory, it is believed that a human host treated with a nutritional package for weight loss and following a regimen of three times a day for three days results in a reduction in weight of at least two pounds, preferably at least six pound, and more preferably at least twelve pounds.

### EXAMPLES

The following examples are provided as actual embodiments of the invention and are not intended to limit the true scope of the invention as pointed out in the claims.

### Example 1

### Preparation of Format Containing Centrum® Tablet

Several Centrum® tablets were crushed into a fine powder. A format was prepared by weighing and mixing together the following nutritional ingredients:

100 mg Centrum®

50 mg Calcium

50 mg B-complex

50 mg Folate (folic acid)

50 mg Niacin

50 mg Niacinamide

Flavored gelatin (6 oz.) was added to the nutritional ingredients. Warm water (2 cups) was then added and the solution stirred until the gelatin dissolved. The solution was poured into a mold and cooled until set.

The product molded firmly with even consistency and coloration. The taste of the format was acceptable.

### Example 2

### Preparation of Format Containing Probiotic and Herbs

A format was prepared by weighing and mixing together the following nutritional ingredients:

100 mg acidophilus powder (capsule crushed into a fine powder)

100 mg Echinacea

100 mg ginkgo biloba

100 mg ginseng extract

100 mg valerian root

100 mg yohimbe

Gelatin was added to the nutritional ingredients. Warm water was then added and the solution stirred until the gelatin dissolved. The solution was poured into a mold, and cooled until set.

The product molded satisfactorily and tasted acceptable.

### Example 3

### Preparation of a Format with Vitamins and Herbs

A format was prepared by weighing and mixing together the following nutritional ingredients:

25 mg thiamine

25 mg riboflavin

25 mg niacin

25 mg vitamin B₆

25 μg folate

25 μg vitamin B₁₂

25 μg biotin

25 mg pantotheric acid

270 mg calcium (as dicalcium phosphate)

300 mg panax ginseng root extract

60 mg gingko biloba extract

100 mg ashwaganolha root extract

Orange flavored gelatin (6 oz) was added to the nutritional ingredients. Warm water (2 cups) was then added and the solution stirred until the gelatin dissolved. The solution was poured into a mold, and cooled until set.

The product molded satisfactorily and tasted acceptable.

While there have been described what are presently believed to be the preferred embodiments of the invention, those skilled in the art will realize that changes and modifications may be made thereto without departing from the spirit of the invention, and it is intended to claim all such changes and modifications as fall within the true scope of the invention.

What is claimed:

1. A format for delivering a desired nutritional package to a human host comprising:
(a) a firm, gelled delivery vehicle;
(b) a package of nutritional ingredients comprising at least one nutritional ingredient selected to provide a human host desired health benefits; and
(c) said nutritional ingredients interspersed and suspended in said delivery vehicle,
whereby a format for delivering a desired nutritional package is provided.

2. A format according to claim 1, wherein said at least one nutritional ingredient is selected from a group consisting of a vitamin, a mineral, an herb, a probiotic, or some combination of said ingredients.

3. A format according to claim 1, wherein said delivery vehicle is selected from the group consisting of gelatin, gum, or combinations thereof.

4. A format according to claim 3, wherein said gelatin is unflavored gelatin.

5. A format according to claim 3, wherein said gelatin is flavored gelatin.

6. A format according to claim 3, wherein said gum is selected from the group consisting of xanthan gum, locust bean gum, guar gum, agar agar gum, and arabic gum.

7. A format according to claim 1, wherein said format provides a nutritional supplement.

8. A format according to claim 1, wherein said health benefit is weight loss.

9. A method of preparing a format for delivery of a desired nutritional package to a human host comprising:
(a) selecting at least one nutritional ingredient for providing a desired health benefit to a human host; and
(b) incorporating said at least one nutritional ingredient in a firm, gelled delivery vehicle,
whereby a format for delivering a desired nutritional package is provided.

10. A method according to claim 9, wherein said at least one nutritional ingredient is selected from the group consisting of a vitamin, a mineral, an herb, a probiotic, or some combination of said ingredients.

11. A method according to claim 9, wherein said delivery vehicle is selected from the group consisting of gelatin, gum, or combinations thereof.

12. A method according to claim 11, wherein the gelatin is unflavored gelatin.

13. A method according to claim 11, wherein said gelatin is flavored gelatin.

14. A method according to claim 11, wherein said gum is selected from the group consisting of xanthan gum, locust bean gum, guar gum, agar agar gum, and arabic gum.

15. A method according to claim 9, wherein said format provides a nutritional supplement.

16. A method according to claim 9, wherein said health benefit is weight loss.

17. A method for treating a human host to provide a desired health benefit comprising administering a nutritional package, comprising at least one nutritional ingredient selected to provide a human host desired health benefit, in a format comprising a firm, gelled delivery vehicle in accordance with a regimen designed to provide said desired health benefit.

18. A method according to claim 17 which further comprises prescribing said regimen for administering said package to provide said health benefit.

19. A method according to claim 17, wherein said regimen comprises a timerelated dosage level for administering said package.

20. A method according to claim 17, wherein said regimen comprises a frequency of administering said package.

21. A method according to claim 17, wherein said regimen comprises a timerelated dosage level and a frequency of administering said package.

22. A method according to claim 17, wherein said package comprises a combination of two or more nutritional ingredients.

23. A method according to claim 17, wherein said desired health benefit is weight loss.

24. A method according to claim 23, wherein said regimen comprises administering said nutritional package formulated for an administration of at least three times a day for three days.

25. A method according to claim 23, wherein said regimen comprises administering said nutritional package formulated for an administration of at least one time a day for two weeks.

26. A method according to claim 17, wherein said at least one nutritional ingredient is selected from a group consisting of a vitamin, a mineral, an herb, a probiotic, or some combination of said ingredients.

27. A method according to claim 17, wherein said delivery vehicle is selected from the group consisting of gelatin, gum, or combinations thereof.

28. A method according to claim 27, wherein the gelatin is unflavored gelatin.

29. A method according to claim 27, wherein said gelatin is flavored gelatin.

30. A method according to claim 27, wherein said gum is selected from the group consisting of xanthan gum, locust bean gum, guar gum, agar agar gum, and arabic gum.

31. A method according to claim 17, wherein said format provides a nutritional supplement.
